(19) United States
(12) Patent Application Publication
(10) Pub. No.: US 2026/0178086 A1
(43) Pub. Date: Jun. 25, 2026

(54) FLEXIBLE PRINTED CIRCUIT BOARD FIXTURE MECHANISMS

(71) Applicant: Oura Health Oy, Oulu (FI)

(72) Inventors: Marko Uusitalo, Oulu (FI); Tuomas Perkkiö, Oulu (FI); Samuel Juho Vertti Poikola, Oulu (FI); Heikki Huttunen, Haukipudas (FI); Pasi Katajamaa, Oulu (FI); Kent Krister Sipola, Oulu (FI)

(21) Appl. No.: 19/427,494

(22) Filed: Dec. 19, 2025

Related U.S. Application Data
(60) Provisional application No. 63/737,298, filed on Dec. 20, 2024.

(51) Int. Cl. G06F 1/16; A61B 5/00

(52) U.S. Cl. CPC G06F 1/1658; A61B 5/0002; A61B 5/6826; A61B 2562/164

(57) ABSTRACT

Methods, systems, and devices for wearable ring device are described. A wearable ring device may include a ring-shaped housing configured to house one or more sensors configured to acquire physiological data from a user, and a flexible printed circuit board (PCB) including electrical circuitry for the one or more sensors. The wearable ring device may include one or more locking grooves disposed within an interior surface of the ring-shaped housing, the one or more locking grooves configured to receive the flexible PCB and maintain a gap between an inner circumferential surface of the ring-shaped housing and a first surface of the flexible PCB.

### CROSS REFERENCE

The present Application for Patent claims the benefit of U.S. Provisional Patent Application No. 63/737,298 by UUSITALO et al., entitled “FLEXIBLE PRINTED CIRCUIT BOARD FIXTURE MECHANISMS,” filed Dec. 20, 2024, assigned to the assignee hereof, and expressly incorporated by reference herein.

### FIELD OF TECHNOLOGY

The following relates to wearable devices and data processing, including flexible printed circuit board (PCB) fixture mechanisms.

### BACKGROUND

Some wearable devices may be configured to measure physiological data from users to help the users understand more about their overall physiological health and well-being. However, wearable devices may be exposed to external forces while worn by the user, which may cause one or more components of the wearable devices to become loose or move in an unintentional manner, which may displace or damage components of the wearable devices, which may further reduce a lifespan of the wearable device.

### BRIEF DESCRIPTION OF THE DRAWINGS

FIGS. 1A, 1B, 1C, and 1D show an example of a device architecture that supports flexible printed circuit board (PCB) fixture mechanisms in accordance with aspects of the present disclosure.

FIGS. 2A, 2B, and 2C show an example of a device architecture that supports flexible PCB fixture mechanisms in accordance with aspects of the present disclosure.

FIG. 3 shows an example of a device architecture that supports flexible PCB fixture mechanisms in accordance with aspects of the present disclosure.

FIGS. 4 and 5 illustrate examples of systems that support flexible PCB fixture mechanisms in accordance with aspects of the present disclosure.

### DETAILED DESCRIPTION

Some wearable devices may be configured to collect data from users associated with movement and other activities. For example, some wearable devices may be configured to continuously acquire physiological data associated with a user including temperature data, heart rate data, and the like. As such, some wearable devices may be configured to house one or more sensors configured to acquire physiological data from a user. In some cases, a wearable device may include a printed circuit board (PCB) including electrical circuitry for the one or more sensors.

In some cases, the PCB may be mounted or otherwise coupled with a housing (e.g., inner housing, outer housing, or both) of the wearable device. For example, in some implementations, the housing may include a cavity in which the PCB is positioned. That is, the PCB may be flexible (e.g., may be a flexible PCB) such that, during a manufacturing process, the PCB may be inserted into the cavity of the housing and may mechanically deform, or flex, from an original shape (e.g., a flat shape) into a curved shape in accordance with a shape of the housing.

In some cases, to secure the PCB in place (e.g., to the housing) and to prevent slippage or movement (e.g., prevent the PCB from returning to an original shape), one or more adhesives (e.g., tapes or glues) may be applied to one or more portions of the PCB to adhere the one or more portions of the PCB to one or more portions of the housing. However, such adhesives or glues may degrade over time (e.g., lose their stickiness), which may result in movement (e.g., shifting or slipping) of the PCB (e.g., may result in the one or more portions of the PCB disconnecting from the housing). Movement of the PCB may further result in misalignment of the PCB with other features of the wearable device. For example, movement of the PCB (e.g., due to one or more forces exerted on the wearable ring device while worn) may result in misalignment of the one or more sensors (e.g., attached to the PCB) with one or more corresponding apertures in the housing, which may result in a decrease in accuracy of measurements collected by the one or more sensors. Additionally, or alternatively, movement of the PCB may cause the PCB to fatigue or break, resulting in the wearable device becoming non-functional.

In accordance with examples described herein, the PCB may include one or more mechanical features that engage (e.g., are inserted into, contact, rub against, apply a friction force to) one or more portions of the housing of the wearable device to secure the PCB in place (e.g., in a desired position, to the housing). In some examples, the one or more mechanical features may be one or more tabs on the PCB that, when inserted into one or more grooves of the housing, secure the PCB in place. That is, a first segment of the PCB may include a first set of tabs and a second segment of the PCB may include a second set of tabs. Similarly, a first segment of the housing may include a first set of grooves and a second segment of the housing may include a second set of grooves, such that, when the PCB is inserted into the housing, the first set of tabs may engage with the first set of grooves and the second set of tabs may engage with the second set of grooves. In some examples, the first segment of the PCB may be associated with a first end of the PCB and the second segment of the PCB may be associated with a second end of the PCB, opposite the first end, or may be associated with a middle segment of the PCB, between the first end and the second end. Additionally, or alternatively, each set of tabs may include one or more first tabs on a first side of the PCB and one or more second tabs on a second side of the PCB, opposite the first side.

In some cases, the PCB may undergo a mechanical deformation during insertion of the PCB into the housing (e.g., to align the one or more tabs on the PCB with the one or more grooves in the housing). That is, in a first shape, the first segment of the PCB and the second segment of the PCB may be of a first width that is wider than a second width of the cavity. As such, during insertion into the cavity, at least a portion of the PCB may mechanically deform, or bend, into a second shape such that the first segment of the PCB fits into the cavity of the housing and, after insertion into the cavity, the PCB may return to the first shape, where the one or more tabs are within, or engage, the one or more grooves in the housing, thus preventing movement of the PCB (e.g., preventing displacement of the PCB greater than a threshold displacement).

Additionally, or alternatively, the housing may include one or more tabs that engage at least a portion of a top surface of (e.g., and apply a friction force to the top of) the PCB when the PCB is inserted into the cavity of the housing, thereby securing the flexible PCB in place. That is, a first portion of the cavity may be of the second width, where the second width is wider than a third width of the PCB. However, one or more second portions of the cavity may include the one or more tabs that restrict the second width of the cavity to a third width of the cavity, where the third width of the cavity is narrower than the third width of the PCB. Thus, during insertion into the cavity, at least a portion of the PCB may deform from the first shape to a third shape such that the portion of the PCB fits within the third width and, after insertion into the cavity, the PCB may return to the first shape, where the top surface of the portion of the PCB contact a bottom surface of the one or more tabs. In such cases, the top surface of the portion of the PCB may exert a force onto the bottom surface of the one or more tabs, which may prevent the PCB from moving unintentionally.

Additionally, or alternatively, the housing may include one or more protrusions (e.g., in the cavity) that engage one or more grooves in the PCB. In other words, the PCB may be inserted into the cavity such that the one or more protrusions engage with the one or more grooves. In such cases, a frictional force may be generated between the one or more protrusions and the one or more grooves during insertion, such that the PCB is prevented from moving unintentionally.

Aspects of the disclosure are initially described in the context of device architectures. Aspects of the disclosure are further described in the context of systems supporting physiological data collection from users via wearable devices,

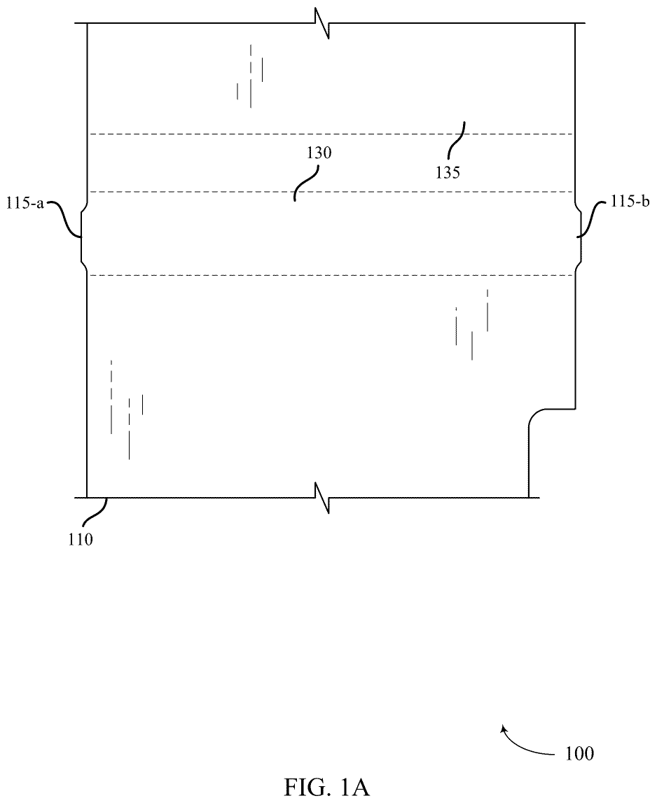

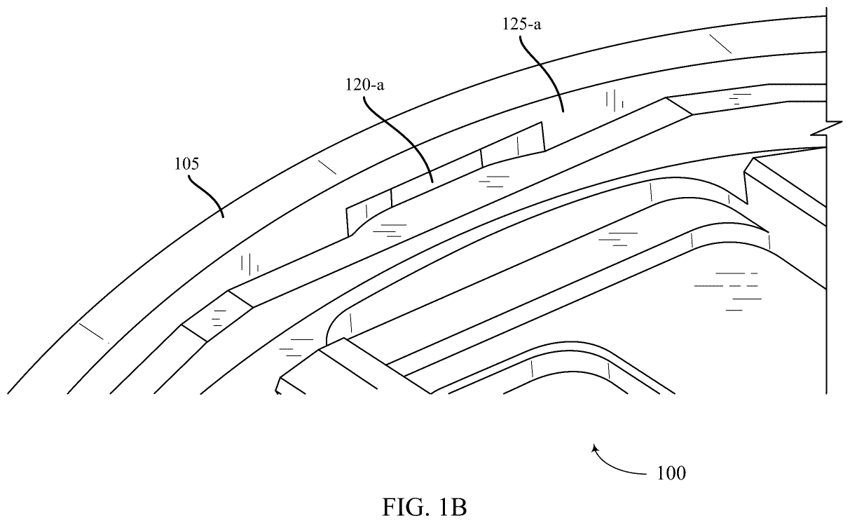

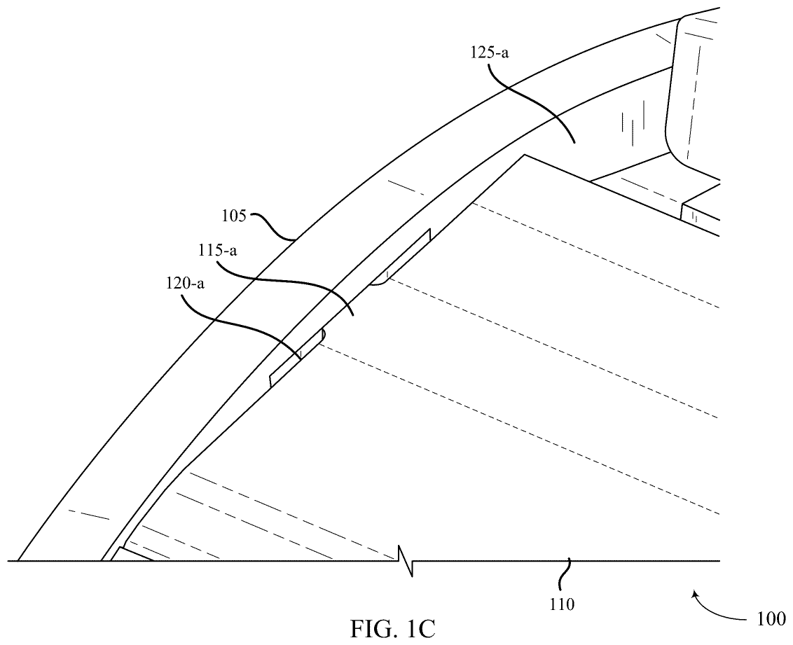

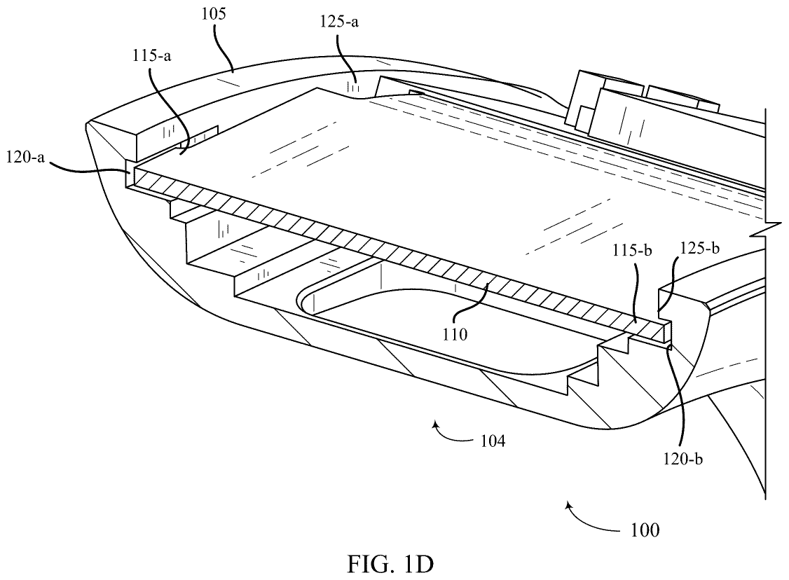

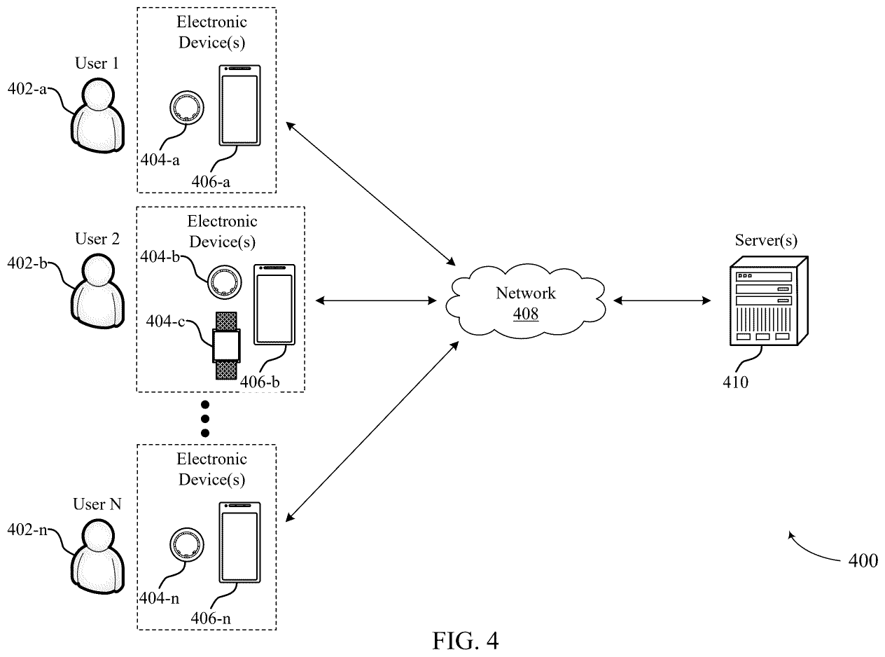

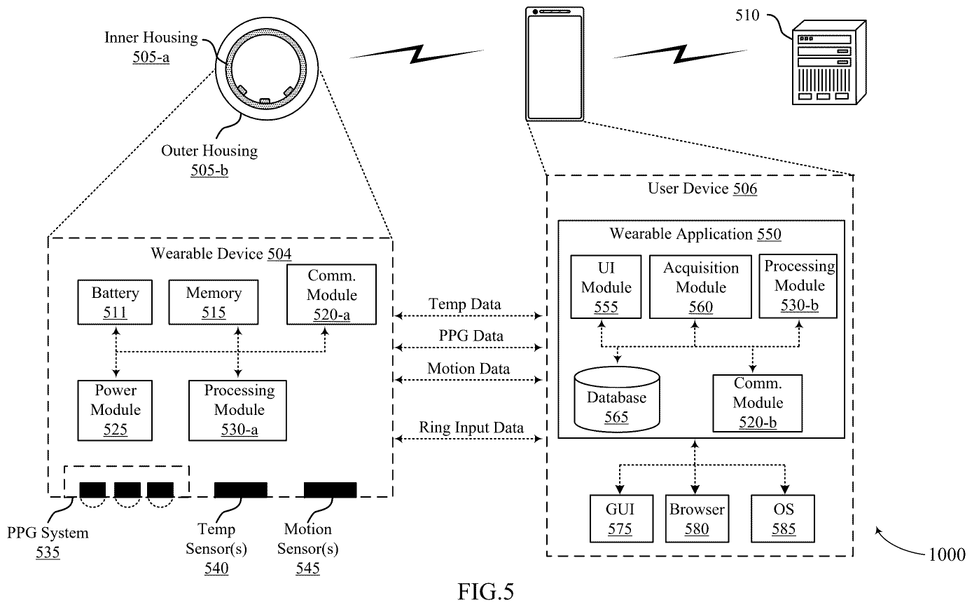

FIGS. 1A, 1B, 1C, and 1D show an example of a device architecture 100 that supports flexible PCB fixture mechanisms in accordance with aspects of the present disclosure. The device architecture 100 may implement or may be implemented by aspects of a system 400, a system 500, or both, as described with reference to FIGS. 4 and 5. For example, the device architecture 100 may be implemented in a ring 104, which may be an example of a wearable device 504, as described with reference to FIG. 5, and may include an inner housing 105, which may be an example of an inner housing 505-a, as described with reference to FIG. 5.

In some examples, the ring 104 may include the inner housing 105 and an outer housing (e.g., such as an outer housing 505-b, as described with reference to FIG. 5), where the inner housing 105 defines an inner circumference of the ring 104 and the outer housing defines an outer circumference of the ring 104. In such cases, the inner housing 105 may be defined by an inner surface (e.g., external facing surface) that faces radially inward and an outer surface that faces radially outward (e.g., faces and at least partially contacts the outer housing).

Additionally, the inner housing 105 may be at least partially hollow (e.g., form a C-shape) and may include a cavity in which a flexible PCB 110 is positioned (e.g., the flexible PCB 110 may be inserted into the cavity during a manufacturing process). The cavity may be at least partially defined by one or more sidewalls 125, including a sidewall 125-a and a sidewall 125-b. In some cases, a design of the the flexible PCB 110 (e.g., a material of the flexible PCB 110, one or more design components of the flexible PCB 110) may enable the flexible PCB 110 to flex, bend, or otherwise deform, such that the flexible PCB 110 may flex to mirror, or otherwise mimic, a curved shape of the inner housing 105. That is, the flexible PCB 110 may undergo a mechanical deformation (e.g., may be flexed into a curved shape) to enable a setting, or positioning, of the flexible PCB 110 into the cavity of the inner housing 105.

In some cases, to secure the flexible PCB 110 in place and to prevent slippage or movement, one or more adhesives (e.g., tapes or glues) may be applied to a bottom surface of the flexible PCB 110 to adhere the flexible PCB 110 to at least a portion of the inner housing 105. However, such adhesives or glues may degrade over time (e.g., lose their stickiness) which may cause the flexible PCB 110 to detach from the inner housing 105 (e.g., compromise a secure placement of the flexible PCB 110), thus allowing the flexible PCB 110 to move (e.g., shift or slip) within the ring 104. Movement of the flexible PCB 110 may result in misalignment of the flexible PCB 110 with other features of the ring 104. For example, movement of the flexible PCB 110 may cause one or more sensors of the ring 104 (e.g., attached to the flexible PCB 110) to become misaligned with one or more corresponding apertures in the inner housing 105, resulting in a decrease in accuracy of measurements collected via the one or more sensors. Additionally, or alternatively, movement of the flexible PCB 110 (e.g., due to external forces caused by a user wearing the ring 104) may cause the flexible PCB 110 to fatigue or break, which may result in the ring 104 being unable to function.

In accordance with examples described herein, the flexible PCB 110 may include one or more mechanical features that engage (e.g., are inserted into, contact, rub against, apply a friction force to) at least a portion of the inner housing 105 to secure the flexible PCB 110 in place (e.g., secure the flexible PCB 110 to the inner housing 105, secure the flexible PCB 110 in a desired positioned). In the example of the device architecture 100, the one or more mechanical features may be one or more tabs 115 (e.g., wings, flanges, extrusions, protrusions). That is, the flexible PCB 110 may include one or more tabs 115 that engage one or more corresponding grooves 120 in the inner housing 105. For example, as depicted in FIG. 1A, a first segment of the flexible PCB 110 may include a first set of tabs 115, including a tab 115-a and a tab 115-b. In some examples, as depicted in FIG. 1A, the tab 115-a and the tab 115-b may be positioned on opposite sides of the flexible PCB 110. That is, the tab 115-a may be positioned on a first side of the flexible PCB 110 and the tab 115-b may be positioned on a second side of the flexible PCB 110, opposite the first side. In some other examples, the first segment may include a single tab 115 on one side of the flexible PCB 110 and the opposite side of the flexible PCB 110 may not include a tab 115. In other words, the flexible PCB 110 may include either the tab 115-a or the tab 115-b in the first segment, but not both. Additionally, or alternatively, the flexible PCB 110 may include one or more additional tabs 115 in the first segment. For example, the first segment of the flexible PCB 110 may include one or more additional tabs on the first side of the flexible PCB 110, the second side of the PCB 110, or both.

It is to be understood that any quantity of tabs 115 or sets (e.g., pairs) of tabs 115 may be implemented at any quantity of segments of or locations on the flexible PCB 110. For example, the flexible PCB 110 may include additional tabs 115 in other segments of the flexible PCB 110. That is, as described herein, FIG. 1A may depict a first segment of the flexible PCB 110, where the first segment of the flexible PCB 110 is located at, or associated with, a first end of the flexible PCB 110. Additionally, a second segment of the PCB 110 may similarly include one or more additional tabs 115 on the first side of the flexible PCB 110, one or more additional tabs 115 on the second side of the flexible PCB 110, or both, where the second segment of the PCB 110 may be located at a second end of the flexible PCB 110 opposite the first end, or at a middle segment of the flexible PCB 110, between the first end and the second end.

As discussed herein, the inner housing 105 may include one or more grooves 120 corresponding to the one or more tabs 115, such that the one or more tabs 115 may mechanically engage the one or more grooves 120. That is, the one or more sidewalls 125 of the cavity of the inner housing 105 may include the one or more grooves 120. For example, as depicted in FIGS. 1B, 1C, and 1D, the sidewall 125-a may include a groove 120-a corresponding to (e.g., configured to engage) the tab 115-a on the flexible PCB 110 and, as depicted in FIG. 1D, the sidewall 125-b may include a groove 120-b corresponding to the tab 115-b on the flexible PCB 110. In some implementations, one of the sidewalls 125 may include one or more grooves 120 and the other sidewall 125 may not include one or more grooves 120. That is, the inner housing 105 may include the groove 120-a or the groove 120-b, but not both. In any case, each groove 120 in the inner housing 105 may correspond to a tab 115 on the flexible PCB 110, such that a quantity of grooves 120 in the inner housing 105 may equal to a quantity of tabs 115 on the flexible PCB 110. For example, continuing with the example described above, the first segment of the flexible PCB 110 may include the tab 115-a and the tab 115-b, such that a first portion of the inner housing 105 may include the groove 120-a and the groove 120-b. Similarly, the second segment of the flexible PCB 110 may include the one or more additional tabs 115 on the first side of the flexible PCB 110, the one or more additional tabs 115 on the second side of the flexible PCB 110, or both, such that a second segment of the inner housing 105 may include one or more additional grooves 120 in the sidewall 125-a, one or more additional grooves in the sidewall 125-b, or both, respectively.

Additionally, as discussed herein, the one or more tabs 115 may be configured to engage (e.g., are inserted into, contact, rub against, apply a friction force to) the one or more grooves 120. For example, during manufacturing, the tab 115-a may be inserted into the groove 120-a and the tab 115-b may be inserted into the groove 120-b, which may secure (e.g., at least partially secure) the flexible PCB 110 in place. To secure the flexible PCB 110 to the inner housing 105, at least a portion of the flexible PCB 110 may deform from a first shape (e.g., original shape, curved shape, undeformed shape) to a second shape (e.g., deformed shape) to enable the one or more tabs 115 to engage the one or more grooves 120. That is, as depicted in FIG. 1A, a portion 130 of the flexible PCB 110 that includes the one or more tabs 115 (e.g., the tab 115-a and the tab 115-b) may be of a first width and a portion 135 of the flexible PCB that does not include the one or more tabs 115 may be of a second width, narrower than the first width. Additionally, the first width may be wider than a third width of an opening of the cavity of the inner housing 105 and the second width may be narrower than the opening of the cavity in the inner housing 105. Thus, to enable the one or more tabs 115 to be inserted within the one or more grooves 120, at least the portion of the flexible PCB 110 may deform such that the portion 130 of the flexible PCB 110 fits within the opening of the cavity of the inner housing 105.

For example, in some cases, the one or more tabs 115 may deform from the first shape to the second shape during insertion of the flexible PCB 110 into the inner housing 105 and may return to the first shape after insertion of the flexible PCB 110 into the inner housing 105. Additionally, or alternatively, the flexible PCB 110 may deform from the first shape to the second shape during insertion of the flexible PCB 110 into the inner housing 105 and may return to the first shape after insertion of the flexible PCB 110 into the inner housing 105. In either case, the one or more tabs 115 may engage (e.g., contact, rub against) the one or more grooves 120 based on at least the portion of the flexible PCB 110 (e.g., the one or more the tabs 115, the flexible PCB 110, or both) returning to the first shape.

Though described in the context of the inner housing 105, this is not to be regarded as a limitation of the present disclosure. In this regard, the inner housing 105 may be interchangeable with the outer housing with regards to the techniques described herein, such that the outer housing may include the one or more grooves 120 that engage the one or more tabs 115 of the flexible PCB 110.

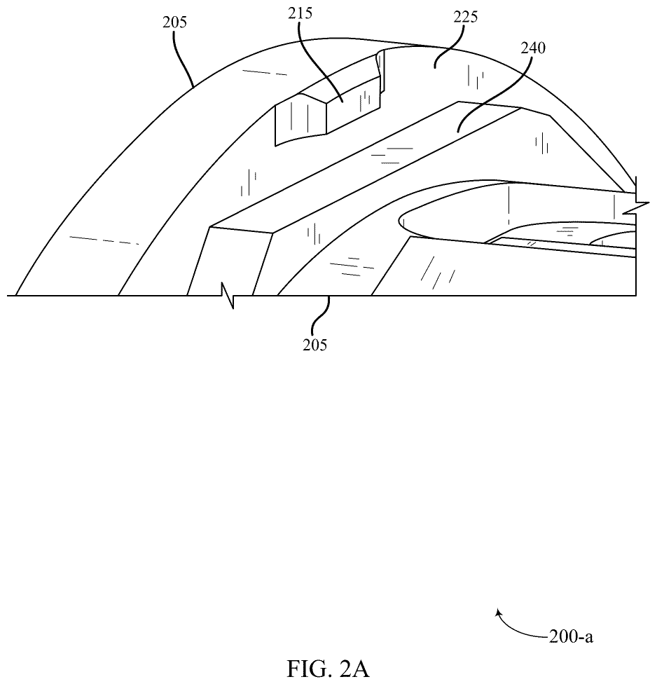

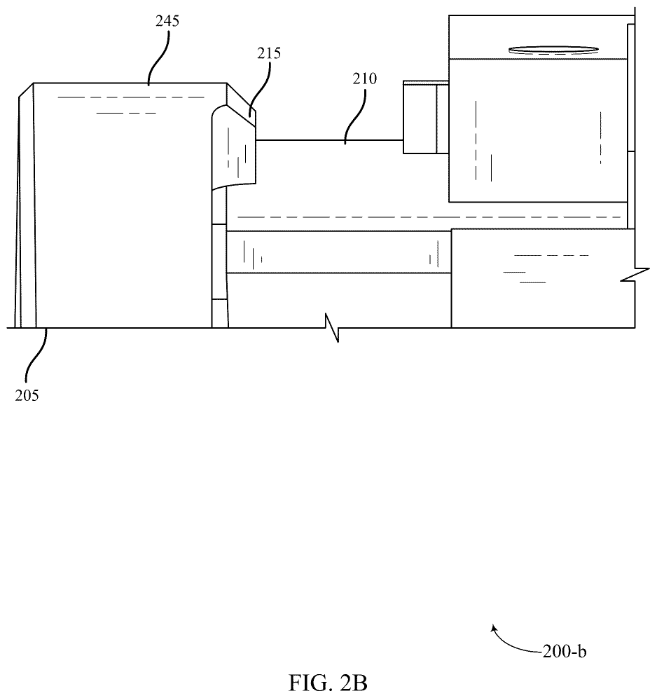

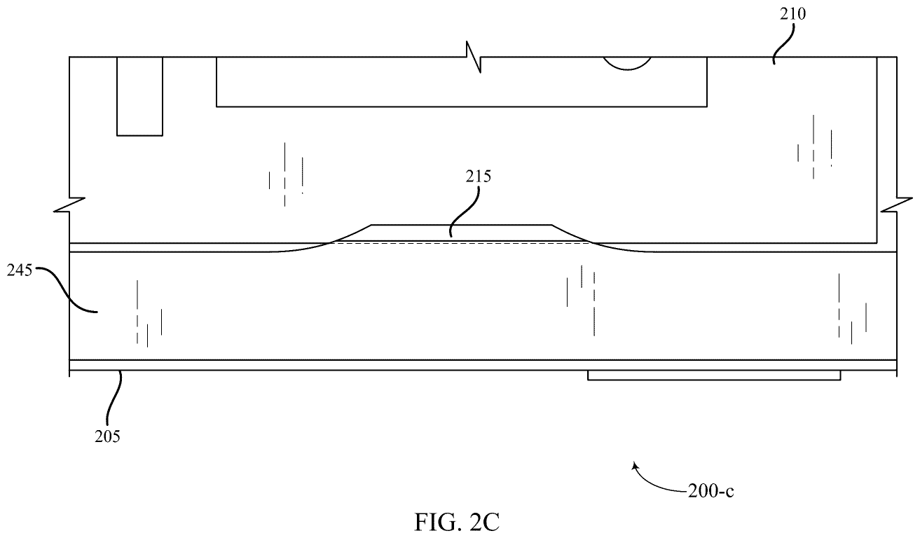

FIGS. 2A, 2B, and 2C show an example of a device architecture 200 that supports flexible PCB fixture mechanisms in accordance with aspects of the present disclosure. The device architecture 200 may implement or may be implemented by aspects of the device architecture 100, the system 400, the system 500, or any combination thereof. For example, the device architecture 200 may be implemented in a ring 104, which may be an example of a wearable device 504, as described with reference to FIG. 5, and may include an inner housing 205, which may be an example of the inner housing 505-a, as described with reference to FIG. 5.

In some examples, the ring 104 may include the inner housing 205 and an outer housing (e.g., such as an outer housing 505-b, as described with reference to FIG. 5), where the inner housing 205 defines an inner circumference of the ring 104 and the outer housing defines an outer circumference of the ring 104. In such cases, the inner housing 205 may be defined by an inner surface (e.g., external facing surface) that faces radially inward and an outer surface (e.g., a surface 245) that faces radially outward (e.g., faces and at least partially contacts the outer housing). As described with reference to FIGS. 1A, 1B, 1C, and 1D, the inner housing 205 may include a cavity in which a flexible PCB 210 is positioned (e.g., inserted), where the cavity is at least partially defined by one or more sidewalls 225 (e.g., a first sidewall 225 and a second sidewall 225).

Additionally, in accordance with examples described herein, the inner housing 205 may include one or more mechanical features that engage (e.g., are inserted into, contact, rub against, apply a friction force to) at least a portion of a flexible PCB 210 to secure the flexible PCB 210 in place. In the example of device architecture 200, the one or more mechanical features may be one or more tabs 215 (e.g., extrusions, lips, flanges). For example, as depicted in FIG. 2A, one or more segments of the inner housing 205 may include one or more tabs 215, where each of the one or more tabs 215 extends from a sidewall 225 of the cavity (e.g., the first sidewall 225 or the second sidewall 225) and, in some cases, may be flush with the surface 245 of the inner housing 205. In such cases, a tab 215 may extend outward from a respective sidewall 225 and may extend over at least a portion of the flexible PCB 210 (e.g., and/or the cavity). Additionally, or alternatively, a bottom of a tab 215 may contact (e.g., rub against, apply a friction force to) a top of the flexible PCB 210, thereby securing (e.g., at least partially securing) the flexible PCB 210 in place.

In some examples, the inner housing 205 may include one or more sets of tabs 215. For example, a set of tabs 215 may include a first tab 215 extending from the first sidewall of the cavity and a second tab 215 extending from the second sidewall of the cavity, opposite the first sidewall. In some other examples, the inner housing 205 may include a single tab 215 on one of the sidewalls 225 and may not include an additional tab 215, opposite the single tab 215, on the other sidewall 225. Additionally, or alternatively, the inner housing 205 may include one or more additional tabs 215 or one or more additional sets of tabs 215 located in one or more other segments of the inner housing 205.

It is to be understood that any quantity of tabs 215 or sets (e.g., pairs) of tabs 215 may be implemented at various segments of or locations on the inner housing 205. For example, a first tab 215 or set of tabs 215 may be positioned in a first segment of the inner housing 205 that is located at (e.g., aligned with) a first end of the flexible PCB 210 and a second tab 215 or set of tabs 215 may be positioned at a second segment of the inner housing 205 that is located at (e.g., aligned with) a second end of the flexible PCB 210 opposite the first end. Additionally, or alternatively, a third tab 215 or set of tabs 215 may be positioned at a third segment of the inner housing 205 that is located at (e.g., aligned with) a middle portion of the flexible PCB 210 between the first end and the second end.

In some examples, to engage the one or more tabs 215 of the inner housing 205, the flexible PCB 210 may deform from a first shape (e.g., original shape, undeformed shaped) to a second shape (e.g., deformed shape) during insertion of the flexible PCB 210 into the inner housing 205 and may return to the first shape after insertion of the flexible PCB 210 into the inner housing 205. That is, a first width of the flexible PCB 210 may be narrower than a second width of the cavity in the inner housing 205, however, the one or more tabs 215 may restrict the second width of the cavity to a third width in one or more portions of the inner housing 205. Thus, to insert the flexible PCB 210 into the cavity, the one or more tabs 215 may exert a force against at least a portion of the flexible PCB 210 during insertion, causing at least the portion of the flexible PCB 210 to bend and eventually pass the one or more tabs 215, returning to the first shape. In some implementations, the one or more tabs 215 of the inner housing 205 may engage (e.g., contact, rub against) the flexible PCB 210 based on the flexible PCB 210 returning to the first shape. Additionally, or alternatively, the flexible PCB 210 may include one or more grooves (e.g., or sets of grooves), and the one or more grooves in the flexible PCB 210 may engage the one or more tabs 215 of the inner housing 205. For example, the one or more tabs 215 of the inner housing 205 may be inserted into the one or more grooves in the flexible PCB 210 during insertion of the flexible PCB 210 into the inner housing 205.

Though described in the context of the inner housing 205, this is not to be regarded as a limitation of the present disclosure. In this regard, the inner housing 205 may be interchangeable with the outer housing with regards to the techniques described herein, such that the outer housing may include the one or more tabs 215 that engage the flexible PCB 210.

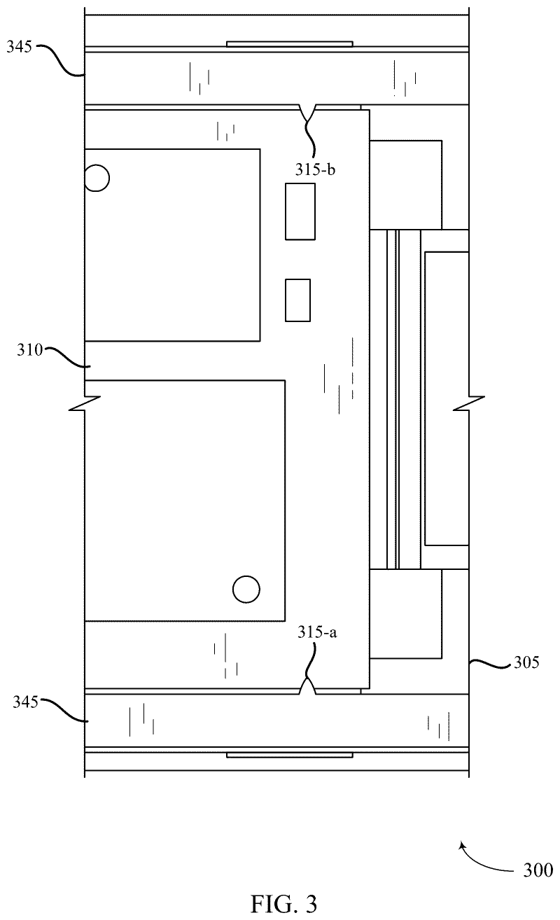

FIG. 3 shows an example of a device architecture 300 that supports flexible PCB fixture mechanisms in accordance with aspects of the present disclosure. The device architecture 300 may implement or may be implemented by aspects of the device architecture 100, the device architecture 200, the system 400, the system 500, or any combination thereof. For example, the device architecture 300 may be implemented in a ring 104, which may be an example of a wearable device 504, as described with reference to FIG. 5, and may include an inner housing 305, which may be an example of the inner housing 505-a as described with reference to FIG. 5.

In some examples, the ring 104 may include the inner housing 305 and an outer housing (e.g., such as an outer housing 505-b, as described with reference to FIG. 5), where the inner housing 305 defines an inner circumference of the ring 104 and the outer housing defines an outer circumference of the ring 104. In such cases, the inner housing 305 may be defined by an inner surface (e.g., external facing surface) that faces radially inward and an outer surface (e.g., a surface 345) that faces radially outward (e.g., faces and at least partially contacts the outer housing). As described with reference to FIGS. 1A, 1B, 1C, and 1D, the inner housing 305 may include a cavity in which a flexible PCB 310 is positioned (e.g., inserted), where the cavity is at least partially defined by one or more sidewalls (e.g., a first sidewall and a second sidewall).

In accordance with examples described herein, the inner housing 305 may include one or more mechanical features which engage (e.g., are inserted into, contact, rub against, apply a friction force to) at least a portion of the flexible PCB 310 to secure the flexible PCB 310 in place. In the example of device architecture 300, the one or more mechanical features may be one or more protrusions 315 that apply a frictional force to at least a portion of the flexible PCB 310. For example, the inner housing 305 may include one or more protrusions 315 in one or more segments of the inner housing 305. In some cases, the one or more protrusions 315 may extend outward from the one or more sidewalls of the cavity and, in some cases, may be flush with the surface 345 of the inner housing 305. For example, a first segment of the inner housing 305 may include a protrusion 315-a and a protrusion 315-b, opposite the protrusion 315-a.

To engage at least a portion of the flexible PCB 310, the one or more protrusions 315 may deform from a first shape (e.g., original shape, undeformed shape) to a second shape after insertion of the flexible PCB 310 into the inner housing 305 and may remain in the second shape after insertion of the flexible PCB 310 into the inner housing 305. In such cases, the one or more protrusions 315 may exert a frictional force on at least the portion of the flexible PCB 310 based on the one or more protrusions 315 remaining in the second shape (e.g., and not returning to the original shape) after insertion of the flexible PCB 310 into the inner housing 305.

In some cases, one or more protrusions 315 may have a triangular shape or may terminate in a point that at least partially contacts the flexible PCB 310. In some examples, the protrusions 315 may engage one or more grooves in the flexible PCB 310 to cause the frictional relationship. Additionally, or alternatively, the protrusions 315 may engage a flat edge of the flexible PCB 310 to cause the frictional relationship. Though illustrated as a single protrusion 315 on either sidewall of the inner housing 305, this is not to be regarded as a limitation of the present disclosure. In this regard, the inner housing 305 may include a set of multiple protrusions 315 positioned (e.g., consecutively) in a row on a same sidewall of the inner housing 305. For example, a set of multiple protrusions 315 may form a ribbed pattern across a segment of the inner housing 305, which may increase a frictional force applied to the flexible PCB 310 relative to a single protrusion 315.

It is to be understood that any quantity of protrusions 315 or sets (e.g., pairs) of protrusions 315 may be implemented at various segments of or locations on the inner housing 305. That is, in some examples, the inner housing 305 may include a single protrusion 315 on the first sidewall and may not include a protrusion 315, opposite the single protrusion 315, on the second sidewall. Additionally, or alternatively, the inner housing 305 may include one or more additional protrusions 315 or pair of protrusions 315 on one or more other segments of the inner housing 305.

Additionally, or alternatively, a first protrusion 315 or set of protrusions 315 may be positioned on a first segment of the inner housing 305 that is located at (e.g., aligned with) a first end of the flexible PCB 310 and a second protrusion 315 or set of protrusions 315 may be positioned at a second segment of the inner housing 305 that is located at (e.g., aligned with) a second end of the flexible PCB 310 opposite the first end. Additionally, or alternatively, a third protrusion 315 or set of protrusions 315 may be positioned at a third segment of the inner housing 305 that is located at (e.g., aligned with) a middle portion of the flexible PCB 310 between the first end and the second end.

Though described in the context of the inner housing 305, this is not to be regarded as a limitation of the present disclosure. In this regard, the inner housing 305 may be interchangeable with the outer housing with regards to the techniques described herein, such that the outer housing may include the one or more protrusions 315 that engage the flexible PCB 310.

FIG. 4 illustrates an example of a system 400 that supports flexible PCB fixture mechanisms in accordance with aspects of the present disclosure. The system 400 includes a plurality of electronic devices (e.g., wearable devices 404, user devices 406) that may be worn and/or operated by one or more users 402. The system 400 further includes a network 408 and one or more servers 410.

The electronic devices may include any electronic devices known in the art, including wearable devices 404 (e.g., ring wearable devices, watch wearable devices, etc.), user devices 406 (e.g., smartphones, laptops, tablets). The electronic devices associated with the respective users 402 may include one or more of the following functionalities: 1) measuring physiological data, 2) storing the measured data, 3) processing the data, 4) providing outputs (e.g., via GUIs) to a user 402 based on the processed data, and 5) communicating data with one another and/or other computing devices. Different electronic devices may perform one or more of the functionalities.

Example wearable devices 404 may include wearable computing devices, such as a ring computing device (hereinafter “ring”) configured to be worn on a user's 402 finger, a wrist computing device (e.g., a smart watch, fitness band, or bracelet) configured to be worn on a user's 402 wrist, and/or a head mounted computing device (e.g., glasses/goggles). Wearable devices 404 may also include bands, straps (e.g., flexible or inflexible bands or straps), stick-on sensors, and the like, that may be positioned in other locations, such as bands around the head (e.g., a forehead headband), arm (e.g., a forearm band and/or bicep band), and/or leg (e.g., a thigh or calf band), behind the ear, under the armpit, and the like. Wearable devices 404 may also be attached to, or included in, articles of clothing. For example, wearable devices 404 may be included in pockets and/or pouches on clothing. As another example, wearable device 404 may be clipped and/or pinned to clothing, or may otherwise be maintained within the vicinity of the user 402. Example articles of clothing may include, but are not limited to, hats, shirts, gloves, pants, socks, outerwear (e.g., jackets), and undergarments. In some implementations, wearable devices 404 may be included with other types of devices such as training/sporting devices that are used during physical activity. For example, wearable devices 404 may be attached to, or included in, a bicycle, skis, a tennis racket, a golf club, and/or training weights.

Much of the present disclosure may be described in the context of a wearable device 404, which may include finger-worn wearable devices, wrist-worn wearable devices, and the like. Accordingly, the terms “wearable device 404,” “wearable ring device,” “ring,” and like terms, may be used interchangeably, unless noted otherwise herein. However, the use of the terms “wearable ring device” and/or “ring” are not to be regarded as limiting, as it is contemplated herein that aspects of the present disclosure may be performed using other wearable devices (e.g., watch wearable devices, necklace wearable device, bracelet wearable devices, earring wearable devices, anklet wearable devices, and the like).

In some aspects, user devices 406 may include handheld mobile computing devices, such as smartphones and tablet computing devices. User devices 406 may also include personal computers, such as laptop and desktop computing devices. Other example user devices 406 may include server computing devices that may communicate with other electronic devices (e.g., via the Internet). In some implementations, computing devices may include medical devices, such as external wearable computing devices (e.g., Holter monitors). Medical devices may also include implantable medical devices, such as pacemakers and cardioverter defibrillators. Other example user devices 406 may include home computing devices, such as internet of things (IoT) devices (e.g., IoT devices), smart televisions, smart speakers, smart displays (e.g., video call displays), hubs (e.g., wireless communication hubs), security systems, smart appliances (e.g., thermostats and refrigerators), and fitness equipment.

Some electronic devices (e.g., wearable devices 404, user devices 406) may measure physiological parameters of respective users 402, such as photoplethysmography waveforms, continuous skin temperature, a pulse waveform, respiration rate, heart rate, heart rate variability (HRV), actigraphy, galvanic skin response, pulse oximetry, blood oxygen saturation (SpO2), blood sugar levels (e.g., glucose metrics), and/or other physiological parameters. Some electronic devices that measure physiological parameters may also perform some/all of the calculations described herein. Some electronic devices may not measure physiological parameters, but may perform some/all of the calculations described herein. For example, a ring (e.g., wearable device 404), mobile device application, or a server computing device may process received physiological data that was measured by other devices.

In some implementations, a user 402 may operate, or may be associated with, multiple electronic devices, some of which may measure physiological parameters and some of which may process the measured physiological parameters. In some implementations, a user 402 may have a ring (e.g., wearable device 404) that measures physiological parameters. The user 402 may also have, or be associated with, a user device 406 (e.g., mobile device, smartphone), where the wearable device 404 and the user device 406 are communicatively coupled to one another. In some cases, the user device 406 may receive data from the wearable device 404 and perform some/all of the calculations described herein. In some implementations, the user device 406 may also measure physiological parameters described herein, such as motion/activity parameters.

For example, as illustrated in FIG. 4, a first user 402-a (User 1) may operate, or may be associated with, a wearable device 404-a (e.g., wearable ring device) and a user device 406-a that may operate as described herein. In this example, the user device 406-a associated with user 402-a may process/store physiological parameters measured by the wearable device 404-a. Comparatively, a second user 402-b (User 2) may be associated with wearable devices 404-b and 404-c (e.g., wearable ring device and a wrist-worn wearable device, such as a watch) and a user device 406-b, where the user device 406-b associated with user 402-b may process/store physiological parameters measured by the wearable devices 404-b and 404-c. Moreover, an nth user 402-n (User N) may be associated with an arrangement of electronic devices described herein (e.g., wearable device 404-n, user device 406-n). In some aspects, wearable devices 404 (e.g., wearable ring devices, wrist-worn wearable devices) and other electronic devices may be communicatively coupled to the user devices 406 of the respective users 402 via Bluetooth, Wi-Fi, and other wireless protocols. Moreover, in some cases, the wearable device 404 and the user device 406 may be included within (or make up) the same device. For example, in some cases, the wearable device 404 may be configured to execute an application associated with the wearable device 404, and may be configured to display data via a GUI.

In some implementations, the wearable devices 404 (e.g., wearable ring devices) of the system 400 may be configured to collect physiological data from the respective users 402 based on arterial blood flow within the user's finger. In particular, a wearable ring device may utilize one or more light-emitting components, such as LEDs (e.g., red LEDs, green LEDs) that emit light on the palm-side of a user's finger to collect physiological data based on arterial blood flow within the user's finger. In general, the terms light-emitting components, light-emitting elements, and like terms, may include, but are not limited to, LEDs, micro LEDs, mini LEDs, laser diodes (LDs) (e.g., vertical cavity surface-emitting lasers (VCSELs), and the like.

In some cases, the system 400 may be configured to collect physiological data from the respective users 402 based on blood flow diffused into a microvascular bed of skin with capillaries and arterioles. For example, the system 400 may collect PPG data based on a measured amount of blood diffused into the microvascular system of capillaries and arterioles. In some implementations, the wearable device 404 may acquire the physiological data using a combination of both green and red LEDs. The physiological data may include any physiological data known in the art including, but not limited to, temperature data, accelerometer data (e.g., movement/motion data), heart rate data, HRV data, blood oxygen level data, or any combination thereof.

The use of both green and red LEDs may provide several advantages over other solutions, as red and green LEDs have been found to have their own distinct advantages when acquiring physiological data under different conditions (e.g., light/dark, active/inactive) and via different parts of the body, and the like. For example, green LEDs have been found to exhibit better performance during exercise. Moreover, using multiple LEDs (e.g., green and red LEDs) distributed around the wearable device 404 (e.g., around an inner surface of the wearable ring device) has been found to exhibit superior performance as compared to wearable devices that utilize LEDs that are positioned close to one another, such as within a watch wearable device. Furthermore, the blood vessels in the finger (e.g., arteries, capillaries) are more accessible via LEDs as compared to blood vessels in the wrist. In particular, arteries in the wrist are positioned on the bottom of the wrist (e.g., palm-side of the wrist), meaning only capillaries are accessible on the top of the wrist (e.g., back of hand side of the wrist), where wearable watch devices and similar devices are typically worn. As such, utilizing LEDs and other sensors within a wearable ring device has been found to exhibit superior performance as compared to wearable devices worn on the wrist, as the wearable ring device may have greater access to arteries (as compared to capillaries), thereby resulting in stronger signals and more valuable physiological data.

The electronic devices of the system 400 (e.g., user devices 406, wearable devices 404) may be communicatively coupled to one or more servers 410 via wired or wireless communication protocols. For example, as shown in FIG. 4, the electronic devices (e.g., user devices 406) may be communicatively coupled to one or more servers 410 via a network 408. The network 408 may implement transfer control protocol and internet protocol (TCP/IP), such as the Internet, or may implement other network 408 protocols. Network connections between the network 408 and the respective electronic devices may facilitate transport of data via email, web, text messages, mail, or any other appropriate form of interaction within a computer network 408. For example, in some implementations, the wearable device 404-a associated with the first user 402-a may be communicatively coupled to the user device 406-a, where the user device 406-a is communicatively coupled to the servers 410 via the network 408. In additional or alternative cases, wearable devices 404 (e.g., wearable ring devices, wrist-worn wearable devices such as watches) may be directly communicatively coupled to the network 408.

The system 400 may offer an on-demand database service between the user devices 406 and the one or more servers 410. In some cases, the servers 410 may receive data from the user devices 406 via the network 408, and may store and analyze the data. Similarly, the servers 410 may provide data to the user devices 406 via the network 408. In some cases, the servers 410 may be located at one or more data centers. The servers 410 may be used for data storage, management, and processing. In some implementations, the servers 410 may provide a web-based interface to the user device 406 via web browsers.

In some aspects, the system 400 may detect periods of time that a user 402 is asleep, and classify periods of time that the user 402 is asleep into one or more sleep stages (e.g., sleep stage classification). For example, as shown in FIG. 4, User 402-a may be associated with a wearable device 404-a (e.g., wearable ring device) and a user device 406-a. In this example, the wearable device 404-a may collect physiological data associated with the user 402-a, including temperature, heart rate, HRV, respiratory rate, and the like. In some aspects, data collected by the wearable device 404-a may be input to a machine learning classifier, where the machine learning classifier is configured to determine periods of time that the user 402-a is (or was) asleep. Moreover, the machine learning classifier may be configured to classify periods of time into different sleep stages, including an awake sleep stage, a rapid eye movement (REM) sleep stage, a light sleep stage (non-REM (NREM)), and a deep sleep stage (NREM). In some aspects, the classified sleep stages may be displayed to the user 402-a via a GUI of the user device 406-a. Sleep stage classification may be used to provide feedback to a user 402-a regarding the user's sleeping patterns, such as recommended bedtimes, recommended wake-up times, and the like. Moreover, in some implementations, sleep stage classification techniques described herein may be used to calculate scores for the respective user, such as Sleep Scores, Readiness Scores, and the like.

In some aspects, the system 400 may utilize circadian rhythm-derived features to further improve physiological data collection, data processing procedures, and other techniques described herein. The term circadian rhythm may refer to a natural, internal process that regulates an individual's sleep-wake cycle, that repeats approximately every 24 hours. In this regard, techniques described herein may utilize circadian rhythm adjustment models to improve physiological data collection, analysis, and data processing. For example, a circadian rhythm adjustment model may be input into a machine learning classifier along with physiological data collected from the user 402-a via the wearable device 404-a. In this example, the circadian rhythm adjustment model may be configured to “weight,” or adjust, physiological data collected throughout a user's natural, approximately 24-hour circadian rhythm. In some implementations, the system may initially start with a “baseline” circadian rhythm adjustment model, and may modify the baseline model using physiological data collected from each user 402 to generate tailored, individualized circadian rhythm adjustment models that are specific to each respective user 402.

In some aspects, the system 400 may utilize other biological rhythms to further improve physiological data collection, analysis, and processing by phase of these other rhythms. For example, if a weekly rhythm is detected within an individual's baseline data, then the model may be configured to adjust “weights” of data by day of the week. Biological rhythms that may require adjustment to the model by this method include: 1) ultradian (faster than a day rhythms, including sleep cycles in a sleep state, and oscillations from less than an hour to several hours periodicity in the measured physiological variables during wake state; 2) circadian rhythms; 3) non-endogenous daily rhythms shown to be imposed on top of circadian rhythms, as in work schedules; 4) weekly rhythms, or other artificial time periodicities exogenously imposed (e.g., in a hypothetical culture with 12 day “weeks,” 12 day rhythms could be used); 5) multi-day ovarian rhythms in women and spermatogenesis rhythms in men; 6) lunar rhythms (relevant for individuals living with low or no artificial lights); and 7) seasonal rhythms.

The biological rhythms are not always stationary rhythms. For example, many women experience variability in ovarian cycle length across cycles, and ultradian rhythms are not expected to occur at exactly the same time or periodicity across days even within a user. As such, signal processing techniques sufficient to quantify the frequency composition while preserving temporal resolution of these rhythms in physiological data may be used to improve detection of these rhythms, to assign phase of each rhythm to each moment in time measured, and to thereby modify adjustment models and comparisons of time intervals. The biological rhythm-adjustment models and parameters can be added in linear or non-linear combinations as appropriate to more accurately capture the dynamic physiological baselines of an individual or group of individuals.

In some aspects, the respective devices of the system 400 may support an apparatus for a wearable device 104 including a locking mechanism for components of the wearable device 104. For example, a wearable ring device (e.g., wearable device 104) may include a ring-shaped housing configured to house one or more sensors configured to acquire physiological data from a user. The wearable ring device (e.g., wearable device 104) may include a flexible PCB including electrical circuitry for the one or more sensors. In some implementations, the wearable ring device 104 may include one or more locking grooves disposed within an interior surface of the ring-shaped housing. The one or more locking grooves may be configured to receive the flexible PCB and maintain a gap between an inner circumferential surface of the ring-shaped housing and a first surface of the flexible PCB.

It should be appreciated by a person skilled in the art that one or more aspects of the disclosure may be implemented in a system 400 to additionally, or alternatively, solve other problems than those described above. Furthermore, aspects of the disclosure may provide technical improvements to “conventional” systems or processes as described herein. However, the description and appended drawings only include example technical improvements resulting from implementing aspects of the disclosure, and accordingly do not represent all of the technical improvements provided within the scope of the claims.

FIG. 5 illustrates an example of a system 500 that supports flexible PCB fixture mechanisms in accordance with aspects of the present disclosure. The system 500 may implement, or be implemented by, system 400. In particular, system 500 illustrates a wearable device 504 (e.g., wearable ring device), a user device 506, and a server 510, as described with reference to FIG. 4.

In some aspects, the wearable device 504 (e.g., wearable ring device) may be configured to be worn around a user's finger, and may determine one or more user physiological parameters when worn around the user's finger. Example measurements and determinations may include, but are not limited to, user skin temperature, pulse waveforms, respiratory rate, heart rate, HRV, blood oxygen levels (SpO2), blood sugar levels (e.g., glucose metrics), and the like.

The system 500 further includes a user device 506 (e.g., a smartphone) in communication with the wearable device 504. For example, the wearable device 504 may be in wireless and/or wired communication with the user device 506. In some implementations, the wearable device 504 may send measured and processed data (e.g., temperature data, photoplethysmogram (PPG) data, motion/accelerometer data, ring input data, and the like) to the user device 506. The user device 506 may also send data to the wearable device 504, such as firmware/configuration updates. The user device 506 may process data. In some implementations, the user device 506 may transmit data to the server 510 for processing and/or storage.

The wearable device 504 may include a housing 505 that may include an inner housing 505-a and an outer housing 505-b. In some aspects, the inner housing 505-a, the outer housing 505-b, or both, may include a curved profile/surface. In particular, the housing 505 may exhibit any curved or “circumferential” profile, including a circular profile, an elliptical profile, and the like. Moreover, in some cases, the inner housing 505-a, the outer housing 505-b, or both, may include both curved (e.g., “circumferential”) and flat/planar portions. For the purposes of the present disclosure, the term “circumferential” may be used interchangeably with the term “curved” to refer to circular-shaped, elliptical-shaped, or other curved-shaped profile.

In some aspects, the housing 505 of the wearable device 504 may store or otherwise include various components of the ring including, but not limited to, device electronics, a power source (e.g., battery 511, and/or capacitor), one or more substrates (e.g., printable circuit boards) that interconnect the device electronics and/or power source, and the like. The device electronics may include device modules (e.g., hardware/software), such as: a processing module 530-a, a memory 515, a communication module 520-a, a power module 525, and the like. The device electronics may also include one or more sensors. Example sensors may include one or more temperature sensors 540, a PPG sensor assembly (e.g., PPG system 535), and one or more motion sensors 545.

The sensors may include associated modules (not illustrated) configured to communicate with the respective components/modules of the wearable device 504, and generate signals associated with the respective sensors. In some aspects, each of the components/modules of the wearable device 504 may be communicatively coupled to one another via wired or wireless connections. Moreover, the wearable device 504 may include additional and/or alternative sensors or other components that are configured to collect physiological data from the user, including light sensors (e.g., LEDs), oximeters, and the like.

The wearable device 504 shown and described with reference to FIG. 5 is provided solely for illustrative purposes. As such, the wearable device 504 may include additional or alternative components as those illustrated in FIG. 5. Additional or alternative wearable devices 504 that provide functionality described herein may be fabricated. For example, wearable devices 504 with fewer components (e.g., sensors) may be fabricated. In a specific example, a wearable device 504 with a single temperature sensor 540 (or other sensor), a power source, and device electronics configured to read the single temperature sensor 540 (or other sensor) may be fabricated. In another specific example, a temperature sensor 540 (or other sensor) may be attached to a user's finger (e.g., using adhesives, wraps, clamps, spring loaded clamps, etc.). In this case, the sensor may be wired to another computing device, such as a wrist worn computing device that reads the temperature sensor 540 (or other sensor). In other examples, a wearable device 504 that includes additional sensors and processing functionality may be fabricated.

The housing 505 may include one or more housing components. The housing 505 may include an outer housing 505-b component (e.g., a shell) and an inner housing 505-a component (e.g., a molding). The housing 505 may include additional components (e.g., additional layers) not explicitly illustrated in FIG. 5. For example, in some implementations, the wearable device 504 may include one or more insulating layers that electrically insulate the device electronics and other conductive materials (e.g., electrical traces) from the outer housing 505-b. The housing 505 may provide structural support for the device electronics, battery 511, substrate(s), and other components. For example, the housing 505 may protect the device electronics, battery 511, and substrate(s) from mechanical forces, such as pressure and impacts. The housing 505 may also protect the device electronics, battery 511, and substrate(s) from water and/or other chemicals.

The inner housing 505-a may be configured to interface with the user's finger. The inner housing 505-a may be formed from a polymer (e.g., a medical grade polymer) or other material. In some implementations, the inner housing 505-a may be transparent. For example, the inner housing 505-a may be transparent to light emitted by the PPG LEDs. In some implementations, the inner housing 505-a component may be molded onto the outer housing 505-b. For example, the inner housing 505-a may include a polymer that is molded (e.g., injection molded) to fit into an outer housing 505-b metallic shell.

The inner housing 505-a and the outer housing 505-b may be fabricated from one or more materials. In some implementations, the inner housing 505-a, the outer housing 505-b, or both, may include a metal, such as titanium, that may provide strength and abrasion resistance at a relatively light weight. Additionally, or alternatively, the inner housing 505-a, and/or the outer housing 505-b may also be fabricated from other materials, such polymers, plastic materials, epoxy materials, ceramic materials, and the like. In some implementations, the outer housing 505-b may be protective as well as decorative.

The wearable device 504 may include one or more substrates (not illustrated). The device electronics and battery 511 may be included on the one or more substrates. For example, the device electronics and battery 511 may be mounted on one or more substrates. Example substrates may include one or more printed circuit boards (PCBs), such as flexible PCB (e.g., polyimide). In some implementations, the electronics/battery 511 may include surface mounted devices (e.g., surface-mount technology (SMT) devices) on a flexible PCB. In some implementations, the one or more substrates (e.g., one or more flexible PCBs) may include electrical traces that provide electrical communication between device electronics. The electrical traces may also connect the battery 511 to the device electronics.

The device electronics, battery 511, and substrates may be arranged in the wearable device 504 in a variety of ways. In some implementations, one substrate that includes device electronics may be mounted along the bottom of the wearable device 504 (e.g., the bottom half), such that the sensors (e.g., PPG system 535, temperature sensors 540, motion sensors 545, and other sensors) interface with the underside of the user's finger. In these implementations, the battery 511 may be included along the top portion of the wearable device 504 (e.g., on another substrate).

The various components/modules of the wearable device 504 represent functionality (e.g., circuits and other components) that may be included in the wearable device 504. Modules may include any discrete and/or integrated electronic circuit components that implement analog and/or digital circuits capable of producing the functions attributed to the modules herein. For example, the modules may include analog circuits (e.g., amplification circuits, filtering circuits, analog/digital conversion circuits, and/or other signal conditioning circuits). The modules may also include digital circuits (e.g., combinational or sequential logic circuits, memory circuits etc.).

The memory 515 (memory module) of the wearable device 504 may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The memory 515 may store any of the data described herein. For example, the memory 515 may be configured to store data (e.g., motion data, temperature data, PPG data) collected by the respective sensors and PPG system 535. Furthermore, memory 515 may include instructions that, when executed by one or more processing circuits, cause the modules to perform various functions attributed to the modules herein. The device electronics of the wearable device 504 described herein are only example device electronics. As such, the types of electronic components used to implement the device electronics may vary based on design considerations.

The functions attributed to the modules of the wearable device 504 (e.g., wearable ring device) described herein may be embodied as one or more processors, hardware, firmware, software, or any combination thereof. Depiction of different features as modules is intended to highlight different functional aspects and does not necessarily imply that such modules must be realized by separate hardware/software components. Rather, functionality associated with one or more modules may be performed by separate hardware/software components or integrated within common hardware/software components.

The processing module 530-a of the wearable device 504 may include one or more processors (e.g., processing units), microcontrollers, digital signal processors, systems on a chip (SOCs), and/or other processing devices. The processing module 530-a communicates with the modules included in the wearable device 504. For example, the processing module 530-a may transmit/receive data to/from the modules and other components of the wearable device 504, such as the sensors. As described herein, the modules may be implemented by various circuit components. Accordingly, the modules may also be referred to as circuits (e.g., a communication circuit and power circuit).

The processing module 530-a may communicate with the memory 515. The memory 515 may include computer-readable instructions that, when executed by the processing module 530-a, cause the processing module 530-a to perform the various functions attributed to the processing module 530-a herein. In some implementations, the processing module 530-a (e.g., a microcontroller) may include additional features associated with other modules, such as communication functionality provided by the communication module 520-a (e.g., an integrated Bluetooth Low Energy transceiver) and/or additional onboard memory 515.

The communication module 520-a may include circuits that provide wireless and/or wired communication with the user device 506 (e.g., communication module 520-b of the user device 506). In some implementations, the communication modules 520-a, 520-b may include wireless communication circuits, such as Bluetooth circuits and/or Wi-Fi circuits. In some implementations, the communication modules 520-a, 520-b can include wired communication circuits, such as Universal Serial Bus (USB) communication circuits. Using the communication module 520-a, the wearable device 504 and the user device 506 may be configured to communicate with each other. The processing module 530-a of the ring may be configured to transmit/receive data to/from the user device 506 via the communication module 520-a. Example data may include, but is not limited to, motion data, temperature data, pulse waveforms, heart rate data, HRV data, PPG data, and status updates (e.g., charging status, battery charge level, and/or wearable device 504 configuration settings). The processing module 530-a of the ring may also be configured to receive updates (e.g., software/firmware updates) and data from the user device 506.

The wearable device 504 may include a battery 511 (e.g., a rechargeable battery 511). An example battery 511 may include a Lithium-Ion or Lithium-Polymer type battery 511, although a variety of battery 511 options are possible. The battery 511 may be wirelessly charged. In some implementations, the wearable device 504 may include a power source other than the battery 511, such as a capacitor. The power source (e.g., battery 511 or capacitor) may have a curved geometry that matches the curve of the wearable device 504. In some aspects, a charger or other power source may include additional sensors that may be used to collect data in addition to, or that supplements, data collected by the wearable device 504 itself. Moreover, a charger or other power source for the wearable device 504 may function as a user device 506, in which case the charger or other power source for the wearable device 504 may be configured to receive data from the wearable device 504, store and/or process data received from the wearable device 504, and communicate data between the wearable device 504 and the servers 510.

In some aspects, the wearable device 504 includes a power module 525 that may control charging of the battery 511. For example, the power module 525 may interface with an external wireless charger that charges the battery 511 when interfaced with the wearable device 504. The charger may include a datum structure that mates with a wearable device 504 datum structure to create a specified orientation with the wearable device 504 during charging. The power module 525 may also regulate voltage(s) of the device electronics, regulate power output to the device electronics, and monitor the state of charge of the battery 511. In some implementations, the battery 511 may include a protection circuit module (PCM) that protects the battery 511 from high current discharge, over voltage during charging, and under voltage during discharge. The power module 525 may also include electro-static discharge (ESD) protection.

The one or more temperature sensors 540 may be electrically coupled to the processing module 530-a. The temperature sensor 540 may be configured to generate a temperature signal (e.g., temperature data) that indicates a temperature read or sensed by the temperature sensor 540. The processing module 530-a may determine a temperature of the user in the location of the temperature sensor 540. For example, in the wearable device 504, temperature data generated by the temperature sensor 540 may indicate a temperature of a user at the user's finger (e.g., skin temperature). In some implementations, the temperature sensor 540 may contact the user's skin. In other implementations, a portion of the housing 505 (e.g., the inner housing 505-a) may form a barrier (e.g., a thin, thermally conductive barrier) between the temperature sensor 540 and the user's skin. In some implementations, portions of the wearable device 504 configured to contact the user's finger may have thermally conductive portions and thermally insulative portions. The thermally conductive portions may conduct heat from the user's finger to the temperature sensors 540. The thermally insulative portions may insulate portions of the wearable device 504 (e.g., the temperature sensor 540) from ambient temperature.

In some implementations, the temperature sensor 540 may generate a digital signal (e.g., temperature data) that the processing module 530-a may use to determine the temperature. As another example, in cases where the temperature sensor 540 includes a passive sensor, the processing module 530-a (or a temperature sensor 540 module) may measure a current/voltage generated by the temperature sensor 540 and determine the temperature based on the measured current/voltage. Example temperature sensors 540 may include a thermistor, such as a negative temperature coefficient (NTC) thermistor, or other types of sensors including resistors, transistors, diodes, and/or other electrical/electronic components.

The processing module 530-a may sample the user's temperature over time. For example, the processing module 530-a may sample the user's temperature according to a sampling rate. An example sampling rate may include one sample per second, although the processing module 530-a may be configured to sample the temperature signal at other sampling rates that are higher or lower than one sample per second. In some implementations, the processing module 530-a may sample the user's temperature continuously throughout the day and night. Sampling at a sufficient rate (e.g., one sample per second) throughout the day may provide sufficient temperature data for analysis described herein.

The processing module 530-a may store the sampled temperature data in memory 515. In some implementations, the processing module 530-a may process the sampled temperature data. For example, the processing module 530-a may determine average temperature values over a period of time. In one example, the processing module 530-a may determine an average temperature value each minute by summing all temperature values collected over the minute and dividing by the number of samples over the minute. In a specific example where the temperature is sampled at one sample per second, the average temperature may be a sum of all sampled temperatures for one minute divided by sixty seconds. The memory 515 may store the average temperature values over time. In some implementations, the memory 515 may store average temperatures (e.g., one per minute) instead of sampled temperatures in order to conserve memory 515.

The sampling rate, which may be stored in memory 515, may be configurable. In some implementations, the sampling rate may be the same throughout the day and night. In other implementations, the sampling rate may be changed throughout the day/night. In some implementations, the wearable device 504 may filter/reject temperature readings, such as large spikes in temperature that are not indicative of physiological changes (e.g., a temperature spike from a hot shower). In some implementations, the wearable device 504 may filter/reject temperature readings that may not be reliable due to other factors, such as excessive motion during exercise (e.g., as indicated by a motion sensor 545).

The wearable device 504 (e.g., communication module) may transmit the sampled and/or average temperature data to the user device 506 for storage and/or further processing. The user device 506 may transfer the sampled and/or average temperature data to the server 510 for storage and/or further processing.

Although the wearable device 504 is illustrated as including a single temperature sensor 540, the wearable device 504 may include multiple temperature sensors 540 in one or more locations, such as arranged along the inner housing 505-a near the user's finger. In some implementations, the temperature sensors 540 may be stand-alone temperature sensors 540. Additionally, or alternatively, one or more temperature sensors 540 may be included with other components (e.g., packaged with other components), such as with the accelerometer and/or processor.

The processing module 530-a may acquire and process data from multiple temperature sensors 540 in a similar manner described with respect to a single temperature sensor 540. For example, the processing module 530 may individually sample, average, and store temperature data from each of the multiple temperature sensors 540. In other examples, the processing module 530-a may sample the sensors at different rates and average/store different values for the different sensors. In some implementations, the processing module 530-a may be configured to determine a single temperature based on the average of two or more temperatures determined by two or more temperature sensors 540 in different locations on the finger.

The temperature sensors 540 on the wearable device 504 (e.g., wearable ring device) may acquire distal temperatures at the user's finger (e.g., any finger). For example, one or more temperature sensors 540 on the wearable device 504 may acquire a user's temperature from the underside of a finger or at a different location on the finger. In some implementations, the wearable device 504 may continuously acquire distal temperature (e.g., at a sampling rate). Although distal temperature measured by a wearable device 504 at the finger is described herein, other devices may measure temperature at the same/different locations. In some cases, the distal temperature measured at a user's finger may differ from the temperature measured at a user's wrist or other external body location. Additionally, the distal temperature measured at a user's finger (e.g., a “shell” temperature) may differ from the user's core temperature. As such, the wearable device 504 may provide a useful temperature signal that may not be acquired at other internal/external locations of the body. In some cases, continuous temperature measurement at the finger may capture temperature fluctuations (e.g., small or large fluctuations) that may not be evident in core temperature. For example, continuous temperature measurement at the finger may capture minute-to-minute or hour-to-hour temperature fluctuations that provide additional insight that may not be provided by other temperature measurements elsewhere in the body.

The wearable device 504 may include a PPG system 535. The PPG system 535 may include one or more optical transmitters that transmit light. The PPG system 535 may also include one or more optical receivers that receive light transmitted by the one or more optical transmitters. An optical receiver may generate a signal (hereinafter “PPG” signal) that indicates an amount of light received by the optical receiver. The optical transmitters may illuminate a region of the user's finger. The PPG signal generated by the PPG system 535 may indicate the perfusion of blood in the illuminated region. For example, the PPG signal may indicate blood volume changes in the illuminated region caused by a user's pulse pressure. The processing module 530-a may sample the PPG signal and determine a user's pulse waveform based on the PPG signal. The processing module 530-a may determine a variety of physiological parameters based on the user's pulse waveform, such as a user's respiratory rate, heart rate, HRV, oxygen saturation, and other circulatory parameters.

In some implementations, the PPG system 535 may be configured as a reflective PPG system 535 where the optical receiver(s) receive transmitted light that is reflected through the region of the user's finger. In some implementations, the PPG system 535 may be configured as a transmissive PPG system 535 where the optical transmitter(s) and optical receiver(s) are arranged opposite to one another, such that light is transmitted directly through a portion of the user's finger to the optical receiver(s).

The number and ratio of transmitters and receivers included in the PPG system 535 may vary. Example optical transmitters may include LEDs. The optical transmitters may transmit light in the infrared spectrum and/or other spectrums. Example optical receivers may include, but are not limited to, photosensors, phototransistors, and photodiodes. The optical receivers may be configured to generate PPG signals in response to the wavelengths received from the optical transmitters. The location of the transmitters and receivers may vary. Additionally, a single device may include reflective and/or transmissive PPG systems 535.

The PPG system 535 illustrated in FIG. 5 may include a reflective PPG system 535 in some implementations. In these implementations, the PPG system 535 may include a centrally located optical receiver (e.g., at the bottom of the wearable device 504) and two optical transmitters located on each side of the optical receiver. In this implementation, the PPG system 535 (e.g., optical receiver) may generate the PPG signal based on light received from one or both of the optical transmitters. In other implementations, other placements, combinations, and/or configurations of one or more optical transmitters and/or optical receivers are contemplated.

The processing module 530-a may control one or both of the optical transmitters to transmit light while sampling the PPG signal generated by the optical receiver. In some implementations, the processing module 530-a may cause the optical transmitter with the stronger received signal to transmit light while sampling the PPG signal generated by the optical receiver. For example, the selected optical transmitter may continuously emit light while the PPG signal is sampled at a sampling rate (e.g., 250 Hz).

Sampling the PPG signal generated by the PPG system 535 may result in a pulse waveform that may be referred to as a “PPG.” The pulse waveform may indicate blood pressure vs time for multiple cardiac cycles. The pulse waveform may include peaks that indicate cardiac cycles. Additionally, the pulse waveform may include respiratory induced variations that may be used to determine respiration rate. The processing module 530-a may store the pulse waveform in memory 515 in some implementations. The processing module 530-a may process the pulse waveform as it is generated and/or from memory 515 to determine user physiological parameters described herein.

The processing module 530-a may determine the user's heart rate based on the pulse waveform. For example, the processing module 530-a may determine heart rate (e.g., in beats per minute) based on the time between peaks in the pulse waveform. The time between peaks may be referred to as an interbeat interval (IBI). The processing module 530-a may store the determined heart rate values and IBI values in memory 515.

The processing module 530-a may determine HRV over time. For example, the processing module 530-a may determine HRV based on the variation in the IBIs. The processing module 530-a may store the HRV values over time in the memory 515. Moreover, the processing module 530-a may determine the user's respiratory rate over time. For example, the processing module 530-a may determine respiratory rate based on frequency modulation, amplitude modulation, or baseline modulation of the user's IBI values over a period of time. Respiratory rate may be calculated in breaths per minute or as another breathing rate (e.g., breaths per 30 seconds). The processing module 530-a may store user respiratory rate values over time in the memory 515.

The wearable device 504 may include one or more motion sensors 545, such as one or more accelerometers (e.g., 6-D accelerometers) and/or one or more gyroscopes (gyros). The motion sensors 545 may generate motion signals that indicate motion of the sensors. For example, the wearable device 504 may include one or more accelerometers that generate acceleration signals that indicate acceleration of the accelerometers. As another example, the wearable device 504 may include one or more gyro sensors that generate gyro signals that indicate angular motion (e.g., angular velocity) and/or changes in orientation. The motion sensors 545 may be included in one or more sensor packages. An example accelerometer/gyro sensor is a Bosch BMI160 inertial micro electro-mechanical system (MEMS) sensor that may measure angular rates and accelerations in three perpendicular axes.

The processing module 530-a may sample the motion signals at a sampling rate (e.g., 50 Hz) and determine the motion of the wearable device 504 based on the sampled motion signals. For example, the processing module 530-a may sample acceleration signals to determine acceleration of the wearable device 504. As another example, the processing module 530-a may sample a gyro signal to determine angular motion. In some implementations, the processing module 530-a may store motion data in memory 515. Motion data may include sampled motion data as well as motion data that is calculated based on the sampled motion signals (e.g., acceleration and angular values).

The wearable device 504 may store a variety of data described herein. For example, the wearable device 504 may store temperature data, such as raw sampled temperature data and calculated temperature data (e.g., average temperatures). As another example, wearable device 504 may store PPG signal data, such as pulse waveforms and data calculated based on the pulse waveforms (e.g., heart rate values, IBI values, HRV values, and respiratory rate values). The wearable device 504 may also store motion data, such as sampled motion data that indicates linear and angular motion.

The wearable device 504, or other computing device, may calculate and store additional values based on the sampled/calculated physiological data. For example, the processing module 530 may calculate and store various metrics, such as sleep metrics (e.g., a Sleep Score), activity metrics, and readiness metrics. In some implementations, additional values/metrics may be referred to as “derived values.” The wearable device 504, or other computing/wearable device, may calculate a variety of values/metrics with respect to motion. Example derived values for motion data may include, but are not limited to, motion count values, regularity values, intensity values, metabolic equivalence of task values (METs), and orientation values. Motion counts, regularity values, intensity values, and METs may indicate an amount of user motion (e.g., velocity/acceleration) over time. Orientation values may indicate how the wearable device 504 is oriented on the user's finger and if the wearable device 504 is worn on the left hand or right hand.

In some implementations, motion counts and regularity values may be determined by counting a number of acceleration peaks within one or more periods of time (e.g., one or more 30 second to 1 minute periods). Intensity values may indicate a number of movements and the associated intensity (e.g., acceleration values) of the movements. The intensity values may be categorized as low, medium, and high, depending on associated threshold acceleration values. METs may be determined based on the intensity of movements during a period of time (e.g., 30 seconds), the regularity/irregularity of the movements, and the number of movements associated with the different intensities.

In some implementations, the processing module 530-a may compress the data stored in memory 515. For example, the processing module 530-a may delete sampled data after making calculations based on the sampled data. As another example, the processing module 530-a may average data over longer periods of time in order to reduce the number of stored values. In a specific example, if average temperatures for a user over one minute are stored in memory 515, the processing module 530-a may calculate average temperatures over a five minute time period for storage, and then subsequently erase the one minute average temperature data. The processing module 530-a may compress data based on a variety of factors, such as the total amount of used/available memory 515 and/or an elapsed time since the wearable device 504 last transmitted the data to the user device 506.

Although a user's physiological parameters may be measured by sensors included on a wearable device 504, other devices may measure a user's physiological parameters. For example, although a user's temperature may be measured by a temperature sensor 540 included in a wearable device 504, other devices may measure a user's temperature. In some examples, other wearable devices (e.g., wrist devices) may include sensors that measure user physiological parameters. Additionally, medical devices, such as external medical devices (e.g., wearable medical devices) and/or implantable medical devices, may measure a user's physiological parameters. One or more sensors on any type of computing device may be used to implement the techniques described herein.

The physiological measurements may be taken continuously throughout the day and/or night. In some implementations, the physiological measurements may be taken during portions of the day and/or portions of the night. In some implementations, the physiological measurements may be taken in response to determining that the user is in a specific state, such as an active state, resting state, and/or a sleeping state. For example, the wearable device 504 can make physiological measurements in a resting/sleep state in order to acquire cleaner physiological signals. In one example, the wearable device 504 or other device/system may detect when a user is resting and/or sleeping and acquire physiological parameters (e.g., temperature) for that detected state. The devices/systems may use the resting/sleep physiological data and/or other data when the user is in other states in order to implement the techniques of the present disclosure.

In some implementations, as described previously herein, the wearable device 504 may be configured to collect, store, and/or process data, and may transfer any of the data described herein to the user device 506 for storage and/or processing. In some aspects, the user device 506 includes a wearable application 550, an operating system 585 (OS), a web browser application (e.g., web browser 580), one or more additional applications, and a GUI 575. The user device 506 may further include other modules and components, including sensors, audio devices, haptic feedback devices, and the like. The wearable application 550 may include an example of an application (e.g., “app”) that may be installed on the user device 506. The wearable application 550 may be configured to acquire data from the wearable device 504, store the acquired data, and process the acquired data as described herein. For example, the wearable application 550 may include a user interface (UI) module 555, an acquisition module 560, a processing module 530-b, a communication module 520-b, and a storage module (e.g., database 565) configured to store application data.

In some cases, the wearable device 504 and the user device 506 may be included within (or make up) the same device. For example, in some cases, the wearable device 504 may be configured to execute the wearable application 550, and may be configured to display data via the GUI 575.

The various data processing operations described herein may be performed by the wearable device 504, the user device 506, the servers 510, or any combination thereof. For example, in some cases, data collected by the wearable device 504 may be pre-processed and transmitted to the user device 506. In this example, the user device 506 may perform some data processing operations on the received data, may transmit the data to the servers 510 for data processing, or both. For instance, in some cases, the user device 506 may perform processing operations that require relatively low processing power and/or operations that require a relatively low latency, whereas the user device 506 may transmit the data to the servers 510 for processing operations that require relatively high processing power and/or operations that may allow relatively higher latency.

In some aspects, the wearable device 504 (e.g., wearable ring device), user device 506, and server 510 of the system 500 may be configured to evaluate sleep patterns for a user. In particular, the respective components of the system 500 may be used to collect data from a user via the wearable device 504, and generate one or more scores (e.g., Sleep Score, Readiness Score) for the user based on the collected data. For example, as noted previously herein, the wearable device 504 of the system 500 may be worn by a user to collect data from the user, including temperature, heart rate, HRV, and the like. Data collected by the wearable device 504 may be used to determine when the user is asleep in order to evaluate the user's sleep for a given “sleep day.” In some aspects, scores may be calculated for the user for each respective sleep day, such that a first sleep day is associated with a first set of scores, and a second sleep day is associated with a second set of scores. Scores may be calculated for each respective sleep day based on data collected by the wearable device 504 during the respective sleep day. Scores may include, but are not limited to, Sleep Scores, Readiness Scores, and the like.

In some cases, “sleep days” may align with the traditional calendar days, such that a given sleep day runs from midnight to midnight of the respective calendar day. In other cases, sleep days may be offset relative to calendar days. For example, sleep days may run from 6:00 pm (18:00) of a calendar day until 6:00 pm (18:00) of the subsequent calendar day. In this example, 6:00 pm may serve as a “cut-off time,” where data collected from the user before 6:00 pm is counted for the current sleep day, and data collected from the user after 6:00 pm is counted for the subsequent sleep day. Due to the fact that most individuals sleep the most at night, offsetting sleep days relative to calendar days may enable the system 500 to evaluate sleep patterns for users in such a manner that is consistent with their sleep schedules. In some cases, users may be able to selectively adjust (e.g., via the GUI) a timing of sleep days relative to calendar days so that the sleep days are aligned with the duration of time that the respective users typically sleep.

In some implementations, each overall score for a user for each respective day (e.g., Sleep Score, Readiness Score) may be determined/calculated based on one or more “contributors,” “factors,” or “contributing factors.” For example, a user's overall Sleep Score may be calculated based on a set of contributors, including: total sleep, efficiency, restfulness, REM sleep, deep sleep, latency, timing, or any combination thereof. The Sleep Score may include any quantity of contributors. The “total sleep” contributor may refer to the sum of all sleep periods of the sleep day. The “efficiency” contributor may reflect the percentage of time spent asleep compared to time spent awake while in bed, and may be calculated using the efficiency average of long sleep periods (e.g., primary sleep period) of the sleep day, weighted by a duration of each sleep period. The “restfulness” contributor may indicate how restful the user's sleep is, and may be calculated using the average of all sleep periods of the sleep day, weighted by a duration of each period. The restfulness contributor may be based on a “wake up count” (e.g., sum of all the wake-ups (when user wakes up) detected during different sleep periods), excessive movement, and a “got up count” (e.g., sum of all the got-ups (when user gets out of bed) detected during the different sleep periods).

The “REM sleep” contributor may refer to a sum total of REM sleep durations across all sleep periods of the sleep day including REM sleep. Similarly, the “deep sleep” contributor may refer to a sum total of deep sleep durations across all sleep periods of the sleep day including deep sleep. The “latency” contributor may signify how long (e.g., average, median, longest) the user takes to go to sleep, and may be calculated using the average of long sleep periods throughout the sleep day, weighted by a duration of each period and the number of such periods (e.g., consolidation of a given sleep stage or sleep stages may be its own contributor or weight other contributors). Lastly, the “timing” contributor may refer to a relative timing of sleep periods within the sleep day and/or calendar day, and may be calculated using the average of all sleep periods of the sleep day, weighted by a duration of each period.

By way of another example, a user's overall Readiness Score may be calculated based on a set of contributors, including: sleep, sleep balance, heart rate, HRV balance, recovery index, temperature, activity, activity balance, or any combination thereof. The Readiness Score may include any quantity of contributors. The “sleep” contributor may refer to the combined Sleep Score of all sleep periods within the sleep day. The “sleep balance” contributor may refer to a cumulative duration of all sleep periods within the sleep day. In particular, sleep balance may indicate to a user whether the sleep that the user has been getting over some duration of time (e.g., the past two weeks) is in balance with the user's needs. Typically, adults need 7-hours of sleep a night to stay healthy, alert, and to perform at their best both mentally and physically. However, it is normal to have an occasional night of bad sleep, so the sleep balance contributor takes into account long-term sleep patterns to determine whether each user's sleep needs are being met. The “resting heart rate” contributor may indicate a lowest heart rate from the longest sleep period of the sleep day (e.g., primary sleep period) and/or the lowest heart rate from naps occurring after the primary sleep period.

Continuing with reference to the “contributors” (e.g., factors, contributing factors) of the Readiness Score, the “HRV balance” contributor may indicate a highest HRV average from the primary sleep period and the naps happening after the primary sleep period. The HRV balance contributor may help users keep track of their recovery status by comparing their HRV trend over a first time period (e.g., two weeks) to an average HRV over some second, longer time period (e.g., three months). The “recovery index” contributor may be calculated based on the longest sleep period. Recovery index measures how long it takes for a user's resting heart rate to stabilize during the night. A sign of a very good recovery is that the user's resting heart rate stabilizes during the first half of the night, at least six hours before the user wakes up, leaving the body time to recover for the next day. The “body temperature” contributor may be calculated based on the longest sleep period (e.g., primary sleep period) or based on a nap happening after the longest sleep period if the user's highest temperature during the nap is at least 0.5° C. higher than the highest temperature during the longest period. In some aspects, the ring may measure a user's body temperature while the user is asleep, and the system 500 may display the user's average temperature relative to the user's baseline temperature. If a user's body temperature is outside of their normal range (e.g., clearly above or below 0.0), the body temperature contributor may be highlighted (e.g., go to a “Pay attention” state) or otherwise generate an alert for the user.

In some aspects, the system 500 may support techniques for fixture of a flexible PCB within the inner housing 505-a or the outer housing 505-b of the ring 104 such that the flexible PCB may not move (e.g., may move less than a threshold amount, or distance)

That is, the flexible PCB may include one or more first mechanical features, as described herein, that engage at least a portion of the inner housing 505-a, the outer housing 505-b, or both. Additionally, or alternatively, the inner housing 505-a, the outer housing 505-b, or both, may include one or more second mechanical features that engage at least a portion of the flexible PCB, the one or more first mechanical features, or both.

It should be noted that the methods described above describe possible implementations, and that the operations and the steps may be rearranged or otherwise modified and that other implementations are possible. Furthermore, aspects from two or more of the methods may be combined.

The following provides an overview of aspects of the present disclosure:

Aspect 1: A wearable ring device, comprising: an outer housing; an inner housing; and a flexible printed circuit board disposed at least partially within the inner housing, the flexible printed circuit board comprising: one or more sensors configured to acquire physiological data; a first mechanical feature on a first segment of the flexible printed circuit board; and a second mechanical feature on a second segment of the flexible printed circuit board, wherein the flexible printed circuit board is configured to undergo a mechanical deformation during insertion into the inner housing, and wherein the first mechanical feature and the second mechanical feature are configured to engage at least a portion of the inner housing based at least in part on the mechanical deformation.

Aspect 2: The wearable ring device of aspect 1, wherein the first mechanical feature comprises a first set of tabs, and the second mechanical feature comprises a second set of tabs.

Aspect 3: The wearable ring device of aspect 2, further comprising: a first set of grooves in the inner housing; and a second set of grooves in the inner housing, wherein the first set of tabs and the second set of tabs are configured to engage the first set of grooves and the second set of grooves, respectively, after the insertion of the flexible printed circuit board into the inner housing.

Aspect 4: The wearable ring device of aspect 3, wherein the first set of tabs and the second set of tabs are configured to: deform from a first shape to a second shape during the insertion of the flexible printed circuit board into the inner housing; and return to the first shape after the insertion of the flexible printed circuit board into the inner housing, wherein the first set of tabs and the second set of tabs are configured to engage the first set of grooves and the second set of grooves, respectively, based at least in part on the return to the first shape.

Aspect 5: The wearable ring device of any of aspects 2 through 4, wherein each of the first set of tabs and the second set of tabs comprise a first tab on a first side of the flexible printed circuit board and a second tab on a second side of the flexible printed circuit board, opposite the first side.

Aspect 6: The wearable ring device of any of aspects 2 through 5, wherein both a first width of the first segment of the flexible printed circuit board and a second width of the second segment of the flexible printed circuit board are wider than a third width of a third segment of the flexible printed circuit board.

Aspect 7: The wearable ring device of aspect 6, wherein both the first width of the first segment of the flexible printed circuit board and the second width of the flexible printed circuit board are wider than a fourth width of a cavity of the inner housing based at least in part on the first set of tabs and the second set of tabs, respectively, and the third width of the third segment of the flexible printed circuit board is narrower than the fourth width of the cavity.

Aspect 8: The wearable ring device of any of aspects 1 through 7, wherein the first segment of the flexible printed circuit board is associated with a first end of the flexible printed circuit board, and the second segment of the flexible printed circuit board is associated with a middle portion of the flexible printed circuit board, between the first end of the flexible printed circuit board and a second end of the flexible printed circuit board.

Aspect 9: The wearable ring device of any of aspects 1 through 8, wherein the first segment of the flexible printed circuit board is associated with a first end of the flexible printed circuit board, and the second segment of the flexible printed circuit board is associated with a second end of the flexible printed circuit board, opposite the first end.

Aspect 10: The wearable ring device of any of aspects 1 through 9, wherein the inner housing comprises a first set of tabs and a second set of tabs.

Aspect 11: The wearable ring device of aspect 10, wherein the first mechanical feature comprises a first set of grooves configured to engage the first set of tabs, and the second mechanical feature comprises a second set of grooves configured to engage the second set of tabs.

Aspect 12: The wearable ring device of any of aspects 1 through 11, wherein the first mechanical feature comprises one or more first protrusions configured to apply a first frictional force to at least a first portion of the inner housing, the second mechanical feature comprises one or more second protrusions, configured to apply a second frictional force to at least a second portion of the inner housing, and the one or more first protrusions and the one or more second protrusions are configured to engage the first portion and the second portion, respectively, of the inner housing, based at least in part on the first frictional force and the second frictional force, respectively.

Aspect 13: The wearable ring device of any of aspects 1 through 12, wherein each of the one or more first protrusions and the one or more second protrusions form a respective ribbed pattern across a respective section of the inner housing, and wherein a respective frictional force is based at least in part on the respective ribbed pattern.

The description set forth herein, in connection with the appended drawings, describes example configurations and does not represent all the examples that may be implemented or that are within the scope of the claims. The term “exemplary” used herein means “serving as an example, instance, or illustration,” and not “preferred” or “advantageous over other examples.” The detailed description includes specific details for the purpose of providing an understanding of the described techniques. These techniques, however, may be practiced without these specific details. In some instances, well-known structures and devices are shown in block diagram form in order to avoid obscuring the concepts of the described examples.

In the appended figures, similar components or features may have the same reference label. Further, various components of the same type may be distinguished by following the reference label by a dash and a second label that distinguishes among the similar components. If just the first reference label is used in the specification, the description is applicable to any one of the similar components having the same first reference label irrespective of the second reference label.

Information and signals described herein may be represented using any of a variety of different technologies and techniques. For example, data, instructions, commands, information, signals, bits, symbols, and chips that may be referenced throughout the above description may be represented by voltages, currents, electromagnetic waves, magnetic fields or particles, optical fields or particles, or any combination thereof.

The various illustrative blocks and modules described in connection with the disclosure herein may be implemented or performed with a general-purpose processor, a DSP, an ASIC, an FPGA or other programmable logic device, discrete gate or transistor logic, discrete hardware components, or any combination thereof designed to perform the functions described herein. A general-purpose processor may be a microprocessor, but in the alternative, the processor may be any conventional processor, controller, microcontroller, or state machine. A processor may also be implemented as a combination of computing devices (e.g., a combination of a DSP and a microprocessor, multiple microprocessors, one or more microprocessors in conjunction with a DSP core, or any other such configuration).

The functions described herein may be implemented in hardware, software executed by a processor, firmware, or any combination thereof. If implemented in software executed by a processor, the functions may be stored on or transmitted over as one or more instructions or code on a computer-readable medium. Other examples and implementations are within the scope of the disclosure and appended claims. For example, due to the nature of software, functions described above can be implemented using software executed by a processor, hardware, firmware, hardwiring, or combinations of any of these. Features implementing functions may also be physically located at various positions, including being distributed such that portions of functions are implemented at different physical locations. Also, as used herein, including in the claims, “or” as used in a list of items (for example, a list of items prefaced by a phrase such as “at least one of” or “one or more of”) indicates an inclusive list such that, for example, a list of at least one of A, B, or C means A or B or C or AB or AC or BC or ABC (i.e., A and B and C). Also, as used herein, the phrase “based on” shall not be construed as a reference to a closed set of conditions. For example, an exemplary step that is described as “based on condition A” may be based on both a condition A and a condition B without departing from the scope of the present disclosure. In other words, as used herein, the phrase “based on” shall be construed in the same manner as the phrase “based at least in part on.”

Computer-readable media includes both non-transitory computer storage media and communication media including any medium that facilitates transfer of a computer program from one place to another. A non-transitory storage medium may be any available medium that can be accessed by a general purpose or special purpose computer. By way of example, and not limitation, non-transitory computer-readable media can comprise RAM, ROM, electrically erasable programmable ROM (EEPROM), compact disk (CD) ROM or other optical disk storage, magnetic disk storage or other magnetic storage devices, or any other non-transitory medium that can be used to carry or store desired program code means in the form of instructions or data structures and that can be accessed by a general-purpose or special-purpose computer, or a general-purpose or special-purpose processor. Also, any connection is properly termed a computer-readable medium. For example, if the software is transmitted from a website, server, or other remote source using a coaxial cable, fiber optic cable, twisted pair, digital subscriber line (DSL), or wireless technologies such as infrared, radio, and microwave, then the coaxial cable, fiber optic cable, twisted pair, DSL, or wireless technologies such as infrared, radio, and microwave are included in the definition of medium. Disk and disc, as used herein, include CD, laser disc, optical disc, digital versatile disc (DVD), floppy disk and Blu-ray disc where disks usually reproduce data magnetically, while discs reproduce data optically with lasers. Combinations of the above are also included within the scope of computer-readable media.

The description herein is provided to enable a person skilled in the art to make or use the disclosure. Various modifications to the disclosure will be readily apparent to those skilled in the art, and the generic principles defined herein may be applied to other variations without departing from the scope of the disclosure. Thus, the disclosure is not limited to the examples and designs described herein, but is to be accorded the broadest scope consistent with the principles and novel features disclosed herein.

What is claimed is:

1. A wearable ring device, comprising:
an outer housing;
an inner housing; and
a flexible printed circuit board disposed at least partially within the inner housing, the flexible printed circuit board comprising:
a first mechanical feature on a first segment of the flexible printed circuit board; and
a second mechanical feature on a second segment of the flexible printed circuit board, wherein the flexible printed circuit board is configured to undergo a mechanical deformation during insertion into the inner housing, and wherein the first mechanical feature and the second mechanical feature are configured to engage at least a portion of the inner housing based at least in part on the mechanical deformation.

2. The wearable ring device of claim 1, wherein the first mechanical feature comprises a first set of tabs, and wherein the second mechanical feature comprises a second set of tabs.

3. The wearable ring device of claim 2, further comprising:
a first set of grooves in the inner housing; and
a second set of grooves in the inner housing, wherein the first set of tabs and the second set of tabs are configured to engage the first set of grooves and the second set of grooves, respectively, after the insertion of the flexible printed circuit board into the inner housing.

4. The wearable ring device of claim 3, wherein the first set of tabs and the second set of tabs are configured to:
deform from a first shape to a second shape during the insertion of the flexible printed circuit board into the inner housing; and
return to the first shape after the insertion of the flexible printed circuit board into the inner housing, wherein the first set of tabs and the second set of tabs are configured to engage the first set of grooves and the second set of grooves, respectively, based at least in part on the return to the first shape.

5. The wearable ring device of claim 2, wherein each of the first set of tabs and the second set of tabs comprise a first tab on a first side of the flexible printed circuit board and a second tab on a second side of the flexible printed circuit board, opposite the first side.

6. The wearable ring device of claim 2, wherein both a first width of the first segment of the flexible printed circuit board and a second width of the second segment of the flexible printed circuit board are wider than a third width of a third segment of the flexible printed circuit board.

7. The wearable ring device of claim 6, wherein both the first width of the first segment of the flexible printed circuit board and the second width of the flexible printed circuit board are wider than a fourth width of a cavity of the inner housing based at least in part on the first set of tabs and the second set of tabs, respectively, and wherein the third width of the third segment of the flexible printed circuit board is narrower than the fourth width of the cavity.

8. The wearable ring device of claim 1, wherein the first segment of the flexible printed circuit board is associated with a first end of the flexible printed circuit board, and wherein the second segment of the flexible printed circuit board is associated with a middle portion of the flexible printed circuit board, between the first end of the flexible printed circuit board and a second end of the flexible printed circuit board.

9. The wearable ring device of claim 1, wherein the first segment of the flexible printed circuit board is associated with a first end of the flexible printed circuit board, and wherein the second segment of the flexible printed circuit board is associated with a second end of the flexible printed circuit board, opposite the first end.

10. The wearable ring device of claim 1, wherein the inner housing comprises a first set of tabs and a second set of tabs.

11. The wearable ring device of claim 10, wherein the first mechanical feature comprises a first set of grooves configured to engage the first set of tabs, and wherein the second mechanical feature comprises a second set of grooves configured to engage the second set of tabs.

12. The wearable ring device of claim 1, wherein the first mechanical feature comprises one or more first protrusions configured to apply a first frictional force to at least a first portion of the inner housing, wherein the second mechanical feature comprises one or more second protrusions, configured to apply a second frictional force to at least a second portion of the inner housing, and wherein the one or more first protrusions and the one or more second protrusions are configured to engage the first portion and the second portion, respectively, of the inner housing, based at least in part on the first frictional force and the second frictional force, respectively.

13. The wearable ring device of claim 12, wherein each of the one or more first protrusions and the one or more second protrusions form a respective ribbed pattern across a respective section of the inner housing, and wherein a respective frictional force is based at least in part on the respective ribbed pattern.

14. A method for manufacturing a wearable ring device, comprising:
inserting a flexible printed circuit board at least partially within an inner housing of the wearable ring device, wherein the flexible printed circuit board undergoes a mechanical deformation during insertion into the inner housing, and wherein a first mechanical feature on a first segment of the flexible printed circuit board engage with a second mechanical feature on a second segment of the flexible printed circuit board based at least in part on the mechanical deformation; and
coupling an outer housing of the wearable ring device with the inner housing of the wearable device to form at least a portion of a frame of the wearable ring device, wherein the outer housing at least partially surrounds the inner housing.

15. The method of claim 14, wherein the first mechanical feature comprises a first set of tabs, and wherein the second mechanical feature comprises a second set of tabs.

16. The method of claim 15, wherein the inner housing comprises a first set of grooves and a second set of grooves, and wherein the first set of tabs and the second set of tabs engage with the first set of grooves and the second set of grooves, respectively, after the insertion of the flexible printed circuit board into the inner housing.

17. The method of claim 15, wherein each of the first set of tabs and the second set of tabs comprise a first tab on a first side of the flexible printed circuit board and a second tab on a second side of the flexible printed circuit board, opposite the first side.

18. The method of claim 15, wherein both a first width of the first segment of the flexible printed circuit board and a second width of the second segment of the flexible printed circuit board are wider than a third width of a third segment of the flexible printed circuit board.

19. The method of claim 19, wherein both the first width of the first segment of the flexible printed circuit board and the second width of the flexible printed circuit board are wider than a fourth width of a cavity of the inner housing based at least in part on the first set of tabs and the second set of tabs, respectively, and wherein the third width of the third segment of the flexible printed circuit board is narrower than the fourth width of the cavity.

20. The method of claim 14, wherein the first segment of the flexible printed circuit board is associated with a first end of the flexible printed circuit board, and wherein the second segment of the flexible printed circuit board is associated with a middle portion of the flexible printed circuit board, between the first end of the flexible printed circuit board and a second end of the flexible printed circuit board.
